(19) United States
(12) Patent Application Publication
(10) Pub. No.: US 2026/0209702 A1
(43) Pub. Date: Jul. 23, 2026

(54) Human Vgamma9Vdelta2T Cell Proliferation Culture Method and Culture Medium

(71) Applicant: GUANGDONG GD KONGMING BIOTECH LLC, Guangzhou City (CN)

(72) Inventors: Zhinan Yin, Guangzhou City (CN); Yangzhe Wu, Guangzhou City (CN); Yan Xu, Guangzhou City (CN)

(21) Appl. No.: 19/571,593

(22) Filed: Mar. 19, 2026

Related U.S. Application Data
(63) Continuation-in-part of application No. 17/418,103, filed on Feb. 19, 2019, now Pat. No. 12,600,945.

(30) Foreign Application Priority Data
Dec. 24, 2018 (CN) 201811580040.2

(51) Int. Cl. C12N 5/0783; A61K 35/17; A61P 35/00

(52) U.S. Cl. CPC C12N 5/0636; A61K 35/17; A61P 35/00; C12N 2500/38; C12N 2500/42; C12N 2500/90; C12N 2501/2302; C12N 2501/2315

(57) ABSTRACT

Provided are a human Vγ9Vδ2T cell proliferation method and a culture medium. The method comprises: first stimulating a human Vγ9Vδ2T cell with a culture medium containing interleukin-2 and phosphonic acid compounds, and then culturing the human Vγ9Vδ2T cell with a culture medium added with interleukin-15 and vitamin C to achieve proliferation culture. The culture medium additionally comprises interleukin-15 and vitamin C. Compared with a conventional proliferation method and a conventional culture medium, the method and the culture medium can improve the proliferation efficiency and cell purity of the human Vγ9Vδ2T cell. The human Vγ9Vδ2T cell obtained by culture in the method has stronger anti-apoptotic ability and longer cell survival time, and moreover, the expression level of critical killer molecules NKG2D thereof is higher, thereby having stronger killing ability to tumor cells.

### CROSS-REFERENCE TO RELATED APPLICATIONS

The present disclosure is a continuation-in-part application of U.S. patent application Ser. No. 17/418,103 filed Jun. 24, 2021, which is a National Stage Application of International Patent Application No. PCT/CN2019/075491, which claims priority to Chinese Patent Application No. 2018115800402, filed with the China National Intellectual Property Administration (CNIPA) on Dec. 24, 2018, and entitled “Human Vγ9Vδ2T Cell Proliferation Culture Method and Culture Medium”, all of which are incorporated herein by reference in their entireties.

### TECHNICAL FIELD

The present disclosure relates to the technical field of cell culture, in particular, to a human Vγ9Vδ2T cell proliferation method and a culture medium.

### BACKGROUND ART

Immunocyte therapy has become a novel important medical method for treating refractory diseases (such as malignant tumor) in the medical field all around the world, for example, a CAR-T cell is used to treat B-cell lymphoma. For the scientific and medical communities, it is very critical to obtain an immunocyte with stable and reliable quality and good functions for rendering good clinical treatment effect.

Human Vγ9Vδ2T cell which only exists in primates and human beings is a T-cell subset with anti-tumor and anti-infection functions. Human Vγ9Vδ2T cell can be used for immunocyte therapy or immunologic function reconstruction of people who suffers from tumor, refractory infectious diseases or immune function unbalanced diseases.

Currently, there are mainly two types of human Vγ9Vδ2T cell proliferation methods:

the first type of existing proliferation technology is to add the monoclonal antibodies of IL-2 and TCR in the culture medium of peripheral blood mononuclear cells (PBMCs). The main disadvantage of the method is that the two types of γδ T cells (Vδ1 cell subset and Vδ2 cell subset) in the peripheral blood are both proliferated, while the Vδ1 cell subset has an immunosuppressive function, and thus it cannot be applied to immunocyte therapy; and
the second type of existing proliferation technology is to add IL-2 and a phosphate small molecule compound such as zoledronic acid in the culture medium of PBMCs. The method can render γδ T cells of the Vδ2 cell subset of a relatively high purity, and thus can be used in immunocyte therapy, and this is a conventional method currently used for proliferating human Vγ9Vδ2T cells in human peripheral blood at home and abroad. However, the method has the following disadvantages: the period of cell culture proliferation is about 14 days, the proliferated human Vγ9Vδ2T cells have a relatively short survival time (which can continuously survive for about 7 days), and thus the cell anti-apoptosis ability is not strong, and thus the ability to kill tumor cells is not strong, either.

### SUMMARY

The purpose of the present disclosure comprises, for example, providing a human Vγ9Vδ2T cell proliferation method, which can improve the proliferation efficiency and the cell purity of human Vγ9Vδ2T cells, and the cultured human Vγ9Vδ2T cells have relatively stronger killing ability and suppressive ability to tumor, and have a stronger anti-apoptotic ability and a longer cell survival time.

The purpose of the present disclosure also comprises, for example, providing a human Vγ9Vδ2T cell culture medium, and the culture medium is used to culture the human Vγ9Vδ2T cells and can improve the proliferation efficiency and the cell purity of the human Vγ9Vδ2T cells, and the cultured human Vγ9Vδ2T cells have a relatively stronger killing ability and suppressive ability to tumor, and have a stronger anti-apoptotic ability and a longer cell survival time.

The purpose of the present disclosure also comprises, for example, providing a human Vγ9Vδ2T cell stimulation and proliferation culture medium, which can selectively proliferate the human Vγ9Vδ2T cells from the peripheral blood mononuclear cells, and the proliferated human Vγ9Vδ2T cells have high purity, stronger killing ability and stronger anti-apoptotic ability.

The present disclosure provides a T cells proliferation method, comprising:
step A, culturing a composition containing the T cells, by using a first culture medium which is able to stimulate the T cells, to stimulate the T cell; and
step B, using a second culture medium to culture the stimulated T cells; wherein the second culture medium contains interleukin-2, interleukin-15, and vitamin C or derivatives of vitamin C.

In one or multiple embodiments, the T cells comprises Vγ9Vδ2T cells.

In one or multiple embodiments, the first culture medium comprises interleukin-2 and phosphonic acid compounds.

In one or multiple embodiments, the first culture medium comprises interleukin-2, phosphonic acid compounds, interleukin-15, and vitamin C or derivatives of vitamin C.

In one or multiple embodiments, the derivatives of vitamin C are selected from vitamin C ethyl ether, vitamin C palmitate, vitamin C glucoside, vitamin C magnesium phosphate and vitamin C sodium phosphate.

In one or multiple embodiments, in the second culture medium, the concentration of the interleukin-15 is 1-1000 ng/ml.

In one or multiple embodiments, in the second culture medium, the concentration of the vitamin C or derivatives of vitamin C is 10 μM-800 mM.

In one or multiple embodiments, in the second culture medium, the concentration of the interleukin-2 is 1-1000 ng/ml.

In one or multiple embodiments, the composition containing the T cells comprises peripheral blood mononuclear cells extracted from the peripheral blood of a human body.

In one or multiple embodiments, in step A, the culture duration is 60-100 hours.

In one or multiple embodiments, the phosphonic acid compounds comprise bisphosphonic acid compounds, preferably, selected from the group consisting of zoledronic acid, etidronic acid, ibandronic acid, pamidronic acid, alendronic acid, risedronic acid, minodronic acid and combinations thereof, for example, zoledronic acid.

In one or multiple embodiments, the second culture medium comprises a basic culture medium, interleukin-2, interleukin-15, and vitamin C or derivatives of vitamin C.

In one or multiple embodiments, the basic culture medium is selected from the group consisting of RPMI-1640 culture medium, D-MEM, MEM, RPMI, Opti-MEM and combinations thereof, for example, RPMI-1640 culture medium.

The present disclosure also provides a culture medium, comprising a basic culture medium, interleukin-2, interleukin-15, and vitamin C or derivatives of vitamin C.

In one or multiple embodiments, the concentration of the interleukin-2 in the culture medium is 1-1000 ng/ml, the concentration of the interleukin-15 is 1-1000 ng/ml, and the concentration of the vitamin C or derivatives of vitamin C is 10 μM-800 mM.

The present disclosure also provides a pharmaceutical composition, comprising the T cells obtained by using the proliferation method according to the present text and a pharmaceutically acceptable carrier.

The present disclosure also provides a pharmaceutical composition, comprising the Vγ9Vδ2T cells obtained by using the proliferation method according to the present text and a pharmaceutically acceptable carrier.

In one or multiple embodiments, the pharmaceutical composition is a cell suspension.

The present disclosure also provides use of the T cells obtained by the proliferation method according to the present disclosure or the pharmaceutical composition of the present disclosure in preparation of medicines for suppressing, preventing or treating infectious diseases, autoimmune diseases or malignant diseases.

In one or multiple embodiments, the malignant disease is cancer.

In one or multiple embodiments, the malignant disease is lung cancer.

The present disclosure also provides a method for suppressing, preventing or treating infectious diseases, autoimmune diseases or malignant diseases, comprising:
(1) extracting peripheral blood mononuclear cells from the peripheral blood of a subject in need;
(2) proliferating the peripheral blood mononuclear cells through the proliferation method of the present disclosure; and
(3) administering the proliferated product to the subject in need.

The present disclosure also provides the use of the T cells obtained by the proliferation method according to the present text or the pharmaceutical composition of the present text in suppressing, preventing or treating infectious diseases, autoimmune diseases or malignant diseases.

The present disclosure also provides the use of the culture medium of the present text used for proliferating T cells.

In one or multiple embodiments, the T cells are Vγ9Vδ2T cells.

The present disclosure provides a human Vγ9Vδ2T cell proliferation method, comprising:
step 1, extracting peripheral blood mononuclear cells from the peripheral blood of a human body;
step 2, providing a first culture medium, resuspending the peripheral blood mononuclear cells by using the first culture medium, to obtain a cell suspension,
wherein the first culture medium comprises a second culture medium and phosphonic acid compounds; and the second culture medium comprises a basic culture medium, and interleukin-2, interleukin-15, and vitamin C or derivatives of vitamin C added in the basic culture medium; and
the order of the step 1 and the step 2 can be adjusted;
step 3 (or step A), inoculating the cell suspension into a cell culture container to culture for 60-100 hours; and
step 4 (or step B), using the second culture medium to change the medium, wherein the second culture medium is used in all the subsequent culture processes.

In one or multiple embodiments, the basic culture medium is the RPMI-1640 culture medium; and the phosphonic acid compounds comprise one or a plurality of zoledronic acid, etidronic acid, ibandronic acid, pamidronic acid, alendronic acid, risedronic acid, minodronic acid.

In one or multiple embodiments, in step 2, the cell density of the cell suspension is 3~4×10⁶ cells/ml.

In one or multiple embodiments, in step 2, in the first culture medium, the concentration of the phosphonic acid compounds is 1-1000 μM; and

In the first culture medium and the second culture medium, the concentration of the interleukin-2 is 1-1000 ng/ml, the concentration of the interleukin-15 is 1-1000 ng/ml, and the concentration of the vitamin C or derivatives of vitamin C is 10 μM-800 mM.

In one or multiple embodiments, the cell culture container is a 24-well plate, 48-well plate or 96-well plate; in the step 3, the cell suspension is inoculated into the cell culture container to culture for 72 hours; and in the subsequent culture process in the step 4, the medium for cells is changed every 48-72 hours.

The present disclosure also provides a culture medium, comprising a basic culture medium, and interleukin-2, interleukin-15, and vitamin C added in the basic culture medium.

In one or multiple embodiments, the basic culture medium is the RPMI-1640 culture medium.

In one or multiple embodiments, in the culture medium, the concentration of the interleukin-2 is 1-1000 ng/ml, the concentration of the interleukin-15 is 1-1000 ng/ml, and the concentration of the vitamin C is 10 μM-800 mM.

The present disclosure also provides a first culture medium, which comprises a second culture medium and phosphonic acid compounds.

In one or multiple embodiments, in the first culture medium, the concentration of the phosphonic acid compounds is 1-1000 μM.

The present disclosure has the following advantageous effects:
(1) compared with a conventional proliferation method (the second type of proliferation technology), the human Vγ9Vδ2T cells obtained by the proliferation method of the present disclosure has a higher proliferation efficiency and a higher cell purity; 90% cell purity can be reached when culturing is conducted for 10-12 days (starting from step 3), and the cell proliferation ratio reaches more than 1000 times (i.e., it can proliferate 100,000 human Vγ9Vδ2T cells to at least 100 million human Vγ9Vδ2T cells), which obviously shortens the proliferation culture period of the human Vγ9Vδ2T cells;
(2) compared with the human Vγ9Vδ2T cells obtained by the conventional proliferation method (the second type of proliferating technology), the human Vγ9Vδ2T cells obtained by the proliferation method of the present disclosure have stronger killing and suppressing abilities to tumor; and
(3) the human Vγ9Vδ2T cells obtained by the proliferation method of the present disclosure have a stronger anti-apoptotic ability and a longer survival time, and after the cell proliferation culture period (10-12 days starting from step 3) ends, the human Vγ9Vδ2T cells obtained by the proliferation method of the present disclosure can continue to survive about 20 days.

### BRIEF DESCRIPTION OF THE DRAWINGS

In order to more clearly illustrate technical solutions of embodiments of the present disclosure, accompanying drawings which need to be used in the embodiments will be introduced briefly below, and it should be understood that the accompanying drawings below merely show some embodiments of the present disclosure, therefore, they should not be considered as limitation to the scope.

FIG. 1 is a comparison chart of the proliferation efficiencies when four different culture mediums are used to conduct proliferation culture to the human Vγ9Vδ2T cells;

FIG. 2 is a comparison chart of the apoptosis ratios of the human Vγ9Vδ2T cells obtained by using four different culture mediums to conduct proliferation culture to the human Vγ9Vδ2T cells;

FIG. 3 is a comparison chart of the expression levels of critical killer molecule NKG2D of the human Vγ9Vδ2T cells obtained by using four different culture mediums to conduct proliferation culture to the human Vγ9Vδ2T cells;

FIG. 4A is a schematic view of the size of tumor after a cell treatment to a humanized mouse with lung cancer using the human Vγ9Vδ2T cells obtained by a conventional proliferation method (IL-2);

FIG. 4B is a schematic view of the size of tumor after a cell treatment to a humanized mouse with lung cancer using the human Vγ9Vδ2T cells obtained by a proliferation method (IL2+IL15+VC) of the present disclosure;

FIG. 4C is a schematic view of the size of tumor of a humanized mouse with lung cancer in the case of not being treated;

FIG. 5 is a schematic view of the variations of the volume of the tumor of humanized mouse with lung cancer under different treating conditions;

FIG. 6 is a schematic view of the variations of the survival rate of humanized mouse with lung cancer under different treating conditions;

FIG. 7 displays the schedule of Vγ9Vδ2 T cell treatments on the patient with cholangiocarcinoma;

FIG. 8 displays the MRI images obtained before entry into the clinical trial and after the 8th treatment course on the patient with cholangiocarcinoma;

FIG. 9 shows the overall survival of patients with advanced liver cancer or lung cancer infused with the Vγ9Vδ2 T cells of the present disclosure;

FIG. 10 shows the expression levels of TNF-α, IFN-γ, CD107a, and NKG2D in the Vγ9Vδ2 T cells of the present disclosure;

FIG. 11 shows the killing effect of the Vγ9Vδ2 T cells of the present disclosure on Huh7 and HepG2 cells;

FIG. 12 is a schematic diagram showing the high-dose tolerance of the Vγ9Vδ2 T cells of the present disclosure;

FIG. 13 is a diagram showing the anti-tumor effect of the Vγ9Vδ2 T cells of the present disclosure in liver cancer-bearing mice; and

FIG. 14 shows the HE and IHC staining results of various internal organs of immunodeficient mice.

### DETAILED DESCRIPTION OF THE EMBODIMENTS

### Terms Used by the Present Disclosure

The term of “basic culture medium” used in the present text refers to a solution containing nutrients that nourish the growth of mammalian cells. The basic culture medium provides standard inorganic salts such as zinc, iron, magnesium, calcium and potassium, and vitamins, glucose, buffer system and essential amino acids. For example, the basic culture medium is RPMI-1640 culture medium, D-MEM, MEM, RPMI, Opti-MEM or the combinations thereof.

The term of “prepared by” has the same meaning with “containing”. The terms of “include”, “comprise”, “have”, “contain” or any other variants used herein are intended to cover non-exclusive comprising. For example, the composition, step, method, article or device comprising listed elements is not necessary to be limited to those elements, other elements not explicitly listed or elements inherent in such a composition, step, method, article or device can also be contained.

The conjunction phrase of “consist of” excludes any unspecified elements, steps or components. If used in a claim, this phrase will make the claim closed, so that the claim does not comprise materials other than those described, except for the conventional impurities associated with them. When the phrase of “consist of” appears in a sub-sentence of the subject of the claim rather than immediately after the subject, it only defines the elements described in the sub-sentence; and other elements are not excluded from the claim as an entirety.

When the amount, concentration, or other value or parameter is expressed by a range, a preferred range, or a range defined by a series of preferred upper limit values and preferred lower limit values, it should be understood as the discourse of all ranges formed by any pairing of any upper limit or preferred value of any range and any lower limit or preferred value of any range, regardless of whether the range is separately disclosed. For example, when the range of “1~5” is disclosed, the described range should be explained to comprise the ranges of “1~4”, “1~3”, “1~2”, “1~2 and 4~5”, “1~3 and 5” and the like. When the value range is described in the present text, unless otherwise described, the range intends to comprise its end values and all integers and fractions within the range.

The term of “parts by mass” refers to the basic unit of measurement that represents the mass ratio relationship of multiple components; 1 part can represent any unit mass, for example, it can represent 1 g, or 2.689 g and the like. If the parts by mass of component A is a parts and the parts by mass of component B is b parts, then it shows that the mass ratio of component A to component B is a:b. Or, it shows that the mass of the component A is aK, and the mass of the component B is bK (K is a random number, representing the multiple factor). What should not be misunderstood is that the sum of the parts by mass of all the components is not limited to 100 parts, which is different from the mass fraction.

The term of “and/or” is used to refer that one or both of the described conditions may occur, for example, A and/or B comprises (A and B) and (A or B).

In addition, the indefinite articles “a” and “an” before an element or component of the present disclosure have no restriction on the quantity (i.e., the occurrence times) of the element or component. Therefore, “a” and “an” should be understood to comprise one or at least one, and the element or component in the singular form also contains the plural form, unless the number described is clearly intended to refer to the singular form.

The term of “derivatives of vitamin C” generally is a type of compounds with an enediol structure that stabilizes a reduced vitamin C by introducing other groups to the carbon atom at position 2 of vitamin C. Examples of derivatives of vitamin C comprise vitamin C phosphate, such as vitamin C magnesium phosphate and vitamin C sodium phosphate; vitamin C palmitate, and vitamin C ethyl ether; and vitamin C carbohydrates, such as vitamin C glucoside and the like.

The terms of “peripheral blood mononuclear cells”, “PBMCs” or “mononuclear cell” refer to mononuclear cells separated from the peripheral blood, and are usually used for anti-cancer immunotherapy. The peripheral blood mononuclear cells can be obtained from collected human blood using, for example, Ficoll-Hypaque density gradient method.

The “peripheral blood mononuclear cells” can be obtained from normal people, subjects who are at risk of having disease, or patients. The peripheral blood mononuclear cells used here need not necessarily be derived from the autologous, and allogeneic peripheral blood mononuclear cells can also be used.

The term of “malignant disease” in the present text is used in the broadest sense thereof, and refers to diseases characterized by uncontrolled cell growth. It comprises, but is not limited to, adrenocortical carcinoma, anal cancer, bladder cancer, ependymoma, medulloblastoma, breast cancer, cervical cancer, colon cancer, endometrial cancer, esophageal cancer, extrahepatic cholangiocarcinoma, eye cancer, gallbladder cancer, gastric cancer, germ cell tumors, extragonadal cancer, head and neck cancer, hypopharyngeal cancer, pancreatic islet cell cancer, laryngeal cancer, leukemia, acute lymphoblastic leukemia, oral cancer, liver cancer, lung cancer, and the like.

The term of “autoimmune disease” refers to diseases and disorders caused by the body's immune response to its own tissues, which causes prolonged inflammation and subsequent tissue destruction. Examples of autoimmune diseases comprise, but is not limited to, alopecia areata, type 1 diabetes, Guillain-Barre syndrome, multiple sclerosis, rheumatoid arthritis, scleroderma, polymyositis, vitiligo, and systemic lupus erythematosus.

The term of “infectious disease” can be the result of any pathogen. Its examples comprise, but is not limited to, the result of viral infections such as AIDS, hepatitis B and C, cell infections, bacterial infections, parasites and fungal infections.

The present disclosure firstly provides a human Vγ9Vδ2T cell proliferation method, comprising:
step 1, extracting peripheral blood mononuclear cells (PBMC) from the peripheral blood of a human body;
step 2, providing a first culture medium, resuspending the peripheral blood mononuclear cells by using the first culture medium, to obtain cell suspension,
wherein the first culture medium comprises a second culture medium and phosphonic acid compounds; and the second culture medium comprises a basic culture medium, and interleukin-2 (IL-2), interleukin-15 (IL-15), and vitamin C added in the basic culture medium; and
the order of the step 1 and the step 2 can be adjusted;
step 3, inoculating the cell suspension into the cell culture container to culture for 60-100 hours; and
step 4, using the second culture medium to change the medium, and using the second culture medium in all the subsequent culture processes.

Specifically, in the step 1, the basic culture medium is RPMI-1640 culture medium. RPMI is short for Roswell Park Memorial Institute, representing Roswell Park Memorial Institute. RPMI is a type of cell culture medium developed by the institute, and 1640 refers to the code name of the culture medium. 10% fetal bovine serum is added when using this culture medium.

Optionally, the phosphonic acid compounds comprise one of or a plurality of zoledronic acid (zoledronate, ZOL), etidronic acid, ibandronic acid, pamidronic acid, alendronic acid, risedronic acid, minodronic acid.

In an embodiment of the present disclosure, the phosphonic acid compound is zoledronic acid.

Specifically, in the step 2, the cell density of the cell suspension is 3~4×10⁶ cells/ml.

Specifically, the purpose of steps 2 and 3 lies in culturing the peripheral blood mononuclear cells by using the first culture medium which contains phosphonic acid compounds, selectively proliferating human Vγ9Vδ2T cells in the peripheral blood mononuclear cells, and suppressing the growing of other cells to make other cells apoptotic.

Optionally, the cell culture container is a 24-well plate, 48-well plate or 96-well plate.

The purpose of the step 4 is to further proliferate the human Vγ9Vδ2T cells with a relatively high purity obtained by the selective proliferation in step 3, to further add the amount of human Vγ9Vδ2T cells.

Specifically, in the step 2, in the first culture medium, the concentration of the phosphonic acid compound is 1-1000 μM; and
in the first culture medium and the second culture medium, the concentration of the interleukin-2 is 1-1000 ng/ml, the concentration of the interleukin-15 is 1-1000 ng/ml, and the concentration of the vitamin C is 10 μM-800 mM.

Preferably, in the step 3, the cell suspension is inoculated into the cell culture container to culture for 72 hours.

Specifically, in the subsequent culture process in the step 4, the medium for cells is changed every 48-72 hours.

Specifically, the total culture duration of the steps 3 and 4 usually is 10-12 days, and the cells at this point reach a sufficient amount and has the strongest killing ability, which are especially suitable for cell therapy.

The present disclosure provides a T cell proliferation method, comprising:
step A, using a first culture medium that comprises interleukin-2 and phosphonic acid compounds, and optionally also comprises interleukin-15, and vitamin C or derivatives of vitamin C to culture a composition containing the T cells to stimulate the T cells; and
step B, using a second culture medium that contains interleukin-2, interleukin-15, and vitamin C or derivatives of vitamin C to culture the stimulated T cells.

The present disclosure provides a human Vγ9Vδ2T cell proliferation method, comprising:
step A, using a culture medium that comprises interleukin-2 and phosphonic acid compounds, and optionally also comprises interleukin-15, and vitamin C or derivatives of vitamin C to culture a composition containing the T cells to stimulate the T cells;
step B, selecting and separating human Vγ9Vδ2T cells from the stimulated peripheral blood mononuclear cells; and
step C, using a second culture medium that contains interleukin-2, interleukin-15, and vitamin C or derivatives of vitamin C to culture the selected and separated human Vγ9Vδ2T cells.

The present disclosure provides a human Vγ9Vδ2T cell proliferation method, comprising:
step A, extracting peripheral blood mononuclear cells (PBMC) from peripheral blood;
step B, using a culture medium which can stimulate T cells to culture a composition containing the T cells to stimulate the T cells;
step C, selecting and separating human Vγ9Vδ2T cells from the stimulated peripheral blood mononuclear cells; and
step D, using a second culture medium that contains interleukin-2, interleukin-15, and vitamin C or derivatives of vitamin C to culture the selected and separated human Vγ9Vδ2T cells.

The present disclosure provides a method for suppressing, preventing or treating infectious diseases, autoimmune diseases or malignant diseases, comprising:
(1) extracting peripheral blood mononuclear cells from the peripheral blood of a first subject;
(2) using a culture medium that contains interleukin-2 and phosphonic acid compounds, and optionally also contains interleukin-15, and vitamin C or derivatives of vitamin C to culture peripheral blood mononuclear cells to stimulate the human Vγ9Vδ2T cells;
(3) using a second culture medium that contains interleukin-2, interleukin-15, and vitamin C or derivatives of vitamin C to culture the stimulated human Vγ9Vδ2T cells;
(4) administering the cultured cells to a second subject, wherein
preferably, the first subject and the second subject can be a same subject. Alternatively, the first subject and the second subject are different subjects.

In the above, the peripheral blood mononuclear cells are compositions containing T cells, and preferably, the malignant disease is cancer, for example, lung cancer.

In the human Vγ9Vδ2T cell proliferation method of the present disclosure, the operating methods without specific conditions are carried out in accordance with the methods described in general conditions (for example, Short Protocols in Molecular Biology, edited by F. M. Ausubel, R. E. Kingston, J. G. Seidman, et al, translated by Ma Xuejun, Shu Yuelong, Beijing: Science Press, 2004).

The critical technical feature of the present disclosure is the use of interleukin-15 and vitamin C in the cell culture process, through adding interleukin-15 and vitamin C in the process of proliferation culture of the human Vγ9Vδ2T cells, compared with the conventional proliferation method (the second type of proliferating technology), the proliferation efficiency and cell purity of the human Vγ9Vδ2T cells can be improved, and the cultured human Vγ9Vδ2T cells have stronger anti-apoptotic ability and longer cell survival time; in addition, the expression level of critical killer molecule NKG2D thereof is higher, and then the killing ability to tumor cells is stronger. Compared with the second type of the existing proliferating technology (referring to the introduction in the background art for the details), the present disclosure solves, for example, the technical problems of low proliferation efficiency and low purity, the problem that the obtained cells do not have a strong killing ability, and the technical problems that the obtained cells do not have enough survival time and the obtained cells are prone to senescence and apoptosis, and the like.

To sum up, the human Vγ9Vδ2T cell proliferation method of the present disclosure has the following advantageous effects:
(1) compared with a conventional proliferation method (the second type of proliferation technology), the human Vγ9Vδ2T cells obtained by the proliferation method of the present disclosure have a better proliferation efficiency and a higher cell purity; 90% cell purity can be reached when the culture is conducted for 10-12 days (starting from step 3), and the cell proliferation ratio reaches more than 1000 times (i.e., it can proliferate 100,000 human Vγ9Vδ2T cells to at least 100 million human Vγ9Vδ2T cells), which obviously shortens the period of proliferation culture for the human Vγ9Vδ2T cells;
(2) compared with the human Vγ9Vδ2T cells obtained by the conventional proliferation method (the second type of proliferating technology), the human Vγ9Vδ2T cells obtained by the proliferation method of the present disclosure have stronger killing and suppressing abilities to tumor; and
(3) the human Vγ9Vδ2T cells obtained by the proliferation method of the present disclosure have a stronger anti-apoptotic ability and a longer cell survival time, and after the cell proliferation culture period (starting from step 3, for 10-12 days) ends, the human Vγ9Vδ2T cells obtained by the proliferation method of the present disclosure can continue surviving for about 20 days.

Based on the above-mentioned human Vγ9Vδ2T cell proliferation method, the present disclosure also provides a human Vγ9Vδ2T cell culture medium (also referred to as the second culture medium in the present text), which comprises a basic culture medium and interleukin-2, interleukin-15, and vitamin C added in the basic culture medium.

In one or multiple embodiments, the basic culture medium is the RPMI-1640 culture medium.

In one or multiple embodiments, in the first culture medium and the second culture medium, the concentration of the interleukin-2 is 1-1000 ng/ml, 1-500 ng/ml, 1-200 ng/ml, or 70-130 ng/ml. In one or multiple embodiments, in the second culture medium, the concentration of the interleukin-15 is 1-1000 ng/ml, 1-500 ng/ml, 1-200 ng/ml, or 70-130 ng/ml. In one or multiple embodiments, in the second culture medium, the concentration of the of the vitamin C is 10 μM-800 mM, or 20 μM-400 mM, or 50 μM-100 μM.

The present disclosure also provides a human Vγ9Vδ2T cell stimulation and proliferation culture medium (also referred to as the first culture medium in the present text), which comprises the above-mentioned second culture medium and phosphonic acid compounds.

Optionally, in the first culture medium, the concentration of the phosphonic acid compound is 1-1000 μM.

### EXAMPLES

### Experimental Reagents and Materials

Main reagents and materials used in the experiments were provided in the following Table 1.

TABLE 1
Main reagents and materials

| | Name of the company & |
|---|---|
| Name of the reagent | producing country |
| DiR dye | Invitrogen, USA |
| CFSE | Sigma, USA |
| DMEM culture medium | Gibco, USA |
| RPMI-1640 culture medium | Gibco, USA |
| Penicillin-streptomycin solution | Gibco, USA |
| PBS | Dongguan Jinan University |
| | Research Institute, China |
| Fetal Bovine Serum | Gibco, USA |
| Dimethyl sulfoxide (DMSO) | Sigma, USA |
| Recombinant Human Interleukin 2 | Beijing Sihuan |
| Injection | Biopharmaceutical |
| | Co., Ltd., China |
| Recombinant human IL-15 | Peprotech, USA |
| L-Ascorbic Acid | Sigma, USA |
| 0.25% Pancreatin | Gibco, USA |
| Zoledronic acid | Sigma, USA |
| Ficoll Lymphocyte Separation | GE Healthcare, USA |
| Solution | |
| Red blood cell lysate | Tiangen Biochemical |
| | Technology |
| | (Beijing) Co., Ltd. |
| Anti-human CD3-BV510 | BD, USA |
| (Clone:SK7) | |
| Anti-human TCR Vδ2-PE (Clone:B6) | BD, USA |
| Anti-human CD314 (NKG2D)- | Biolegend, USA |
| PerCP/Cy5.5 (Clone:1D11) | |
| Anti-human Ki67-Alexa Fluor ® 647 | BD, USA |
| (Clone: B56) | |
| Anti-human CD45RA-PE-cy5 | Biolegend, USA |
| (Clone:HI100) | |
| Anti-human CD27-Pacific Blue | Biolegend, USA |
| (Clone: O323) | |

### Example 1: Vγ9Vδ2T Cell Proliferation Effects and Characteristics of Vγ9Vδ2T Cells Obtained by Proliferation

### Extraction of Peripheral Blood Mononuclear Cells

1) 4 mL of human lymphocyte separation solution was taken and added into a 15 ml conical centrifuge tube, 1×PBS buffer was used to dilute the peripheral blood in equal proportions and mixed thoroughly, and then 10 ml of diluted peripheral blood was slowly spread on the surface of the lymphocyte separation solution along the wall of the test tube, and 600 g centrifuging was conducted for 25 min at 25° C.
2) A sterile Pasteur pipette was used to draw the middle white flocculent cell layer into another sterile centrifuge tube, an equal volume of PBS was added and mixed thoroughly, and then centrifuging was conducted at 1500 rpm for 10 min.
3) After the supernatant was discarded, 5~10 mL red blood cell lysate buffer was added to resuspend the cells to lyse for 3~7 min at room temperature. 5 times of the volume of PBS was added to terminating the lysis. After filtering with a 40 μm screen, centrifuging was conducted at 1500 rpm for 10 min.
4) After the supernatant was discarded, 10 mL of serum-free RPMI1640 culture medium was added to resuspend the cells, and centrifuging was conducted at 1500 rpm for 10 min.
5) After the supernatant was discarded, the RPMI1640 complete culture medium (containing 10% fetal bovine serum) was used to dilute the cells sinking at the bottom of the tube to 2 mL, after mixing thoroughly and slowly, 5 μL of cell diluent was drawn for diluting to an appropriate multiple, and a hemocytometer was used for counting.

### Proliferation In Vitro of Vγ9Vδ2T Cell

After the PBMC in fresh blood was separated using the Ficoll separation solution, RPMI-1640 complete culture medium with 10% FBS was used to adjust the cell concentration to 3~4×10⁶ cells/ml, and inoculated to a 24-well plate, and 40 ng/mL IL-2 and 50 μM zoledronic acid were added for stimulating for 3 days. Then, the zoledronic acid was removed, the medium was changed every 2~3 days for passage, and the RPMI-1640 culture medium containing cytokines of the following concentrations was used: 100 IU/mL IL-15, 100 IU/mL IL-2, and 70 μM vitamin C, to culture in a culture incubator at 37° C., 5% CO₂, PH=7.2-7.4, and a humidity of 95% for 10-14 days.

### Vγ9Vδ2T Cell Purity and Phenotype Identification

Vγ9Vδ2T cells proliferated in vitro for 10~14 days were collected and placed inside a 1.5 mL of EP tube, and then centrifuged at 3500 rpm for 5 min in a miniature centrifuge (Eppendorf 5424), and after the supernatant was discarded, washing was conducted twice with 4° C. pre-cooled PBS; the cells were transferred into the corresponding test tubes according to the blank control tube, isotype control and experimental group, respectively, and each tube contained about 5×10⁵ cells. The control tube and the detection tube were respectively equipped with the following fluorescent antibody staining solution according to the antibody specification: anti-human CD3-V500, anti-human TCR-δ2-PE, anti-human CD45-PE-cy5, anti-human CD27-PB and NKG2D-PE-cy7. The cells were resuspended with the prepared fluorescent antibody staining solution, then placed in a refrigerator at 4° C. or on ice, stained in a dark place for 15-20 minutes, washed twice with PBS, and centrifuged at 3500 rpm for 5 minutes, and the supernatant was discarded, and then the cells were resuspended with 200-300 μL PBS, and the purity and phenotype of Vγ9Vδ2T cells were detected using a flow cytometry.

Vγ9Vδ2T Cell ki67 Assay
1) 7~10×10⁵ Vγ9Vδ2T cells proliferated in vitro for 10~14 days were collected, placed inside a 1.5 mL EP tube, centrifuged at 3500 rpm for 5 min in a miniature centrifuge (Eppendorf 5424), and after discarding the supernatant, washed twice using 4° C. pre-cooled PBS.
2) The cell pellet was resuspended by 100 μL PBS, surface-stained flow cytometry antibody CD3-FITC and TCR-δ2-PE (1:200 dilution) were added, an isotype control antibody of the same color and same concentration was added to the control tube, and they were incubated at 4° C. in the dark for 15 minutes.
3) After staining, the resultant was washed twice in PBS, and centrifuged at 3500 rpm for 5 min.
4) Foxp3 Staining Buffer Set was used to detect the expression level of intra-nuclear ki67 as follows: preparing Fixation & Permeabilization Buffer, evenly mixing F & P Concentration with F & P Diluent in a volume ratio of 1:3; and resuspending the cells with 500 μL F & P Buffer, standing in a refrigerator at 4° C. or on ice in dark place for 0.5-18 hours, thereby fixing and permeabilizing the cells.
5) The 10×Permeabilization Buffer was diluted to 1×Permeabilization Buffer with ddH₂O. After fixing, 1 mL 1×Permeabilization Buffer was added, and the resultant was centrifuged at 5500 rpm at 4° C. for 5 min and washed twice.
6) The APC-Ki67 fluorescent antibody was diluted into 1×Perm Buffer in a volume ratio of 1:200 and mixed evenly; the cell pellet was resuspended with a staining solution, placed in a refrigerator at 4° C. or on ice, and stained in dark place for 30 minutes.
7) 1×Perm Buffer solution was added, the resultant was centrifuged at 5500 rpm at 4° C. for 5 min, and resuspended with 1×Perm Buffer solution and centrifuged again to wash away unbound fluorescent antibody. The cells were resuspended with 200-300 μL PBS, the volume was adjusted, and the Ki67 expression level was analyzed with a flow cytometry.

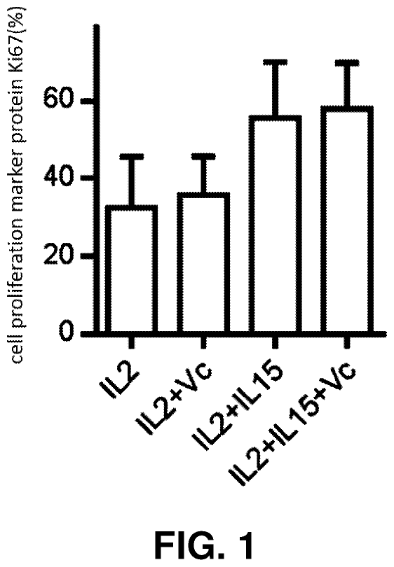

FIG. 1 was a comparison chart of the proliferation ratios showing that a culture medium containing zoledronic acid was first used for stimulation and then four different cell culture mediums were used to conduct proliferation culture to the human Vγ9Vδ2T cells.

In FIG. 1, IL2 indicated what was used in the entire proliferation culture process was the culture medium obtained by adding IL2 based on the basic culture medium;

IL2+VC indicated what was used in the entire proliferation culture process was the culture medium obtained by adding IL2+VC based on the basic culture medium;

IL2+IL15 indicated what was used in the entire proliferation culture process was the culture medium obtained by adding IL2+IL15 based on the basic culture medium; and

IL2+IL15+VC indicated what was used in the entire proliferation culture process was the culture medium obtained by adding IL2+IL15+VC based on the basic culture medium.

In the stage of stimulation and proliferation, the solutions of the above four culture mediums all used the basic culture medium added with ZOL (zoledronic acid) and IL-2 to stimulate the cells, and the amount of the components of each culture medium in FIG. 1 refers to the concentration as described in the portion of “Vγ9Vδ2T cell proliferation in vitro” of this example.

As shown in FIG. 1, after the above four culture mediums were respectively used for conducting proliferation culture to the human Vγ9Vδ2T cells, the obtained data showed that the cell proliferation efficiency of the culture medium (IL2 and IL2+VC), in which IL15 was not added, was relatively low, while the cell proliferation efficiency of the culture medium (IL2+IL15 and IL2+IL15+VC), in which IL15 was added, was relatively high, and thus, it could be seen that the adding of IL-15 could obviously improve the proliferation efficiency (rise of Ki67 protein expression) of the human Vγ9Vδ2T cells.

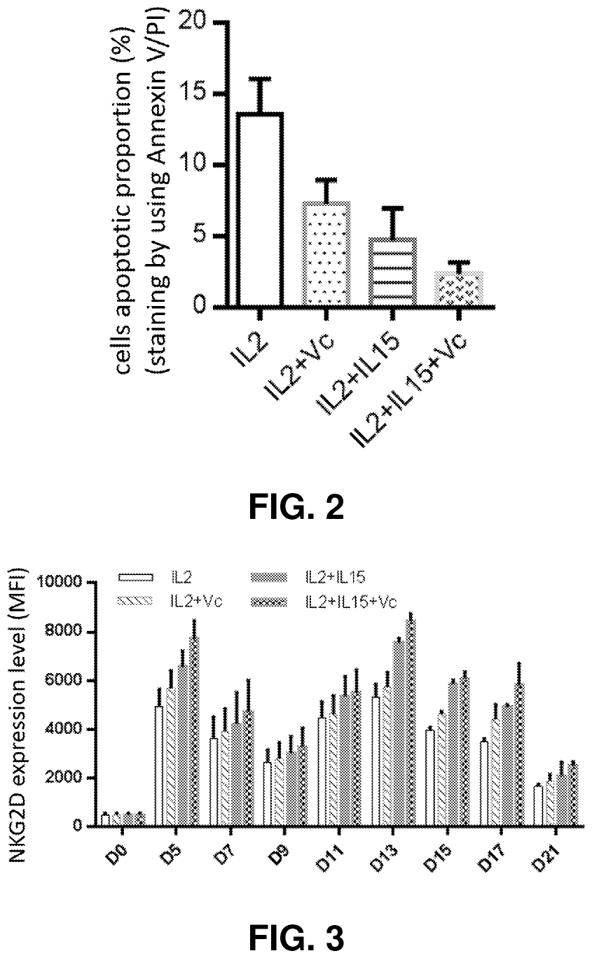

FIG. 2 was a comparison chart of the apoptosis ratio of the human Vγ9Vδ2 T cells obtained by using four different culture mediums to conduct proliferation culture to the human Vγ9Vδ2T cells, and IL2, IL2+VC, IL2+IL15, IL2+IL 15+VC in FIG. 2 represented the same meanings with those in FIG. 1.

Based on FIG. 2, it could be seen that adding VC into the culture medium could obviously reduce the apoptosis ratio and apoptosis rate of cells, and in addition, the combination of IL-15 and VC could more significantly enhance the anti-apoptotic ability of cells.

FIG. 3 was a comparison chart of the expression levels of the critical killer molecule NKG2D of the human Vγ9Vδ2 T cells obtained by using four different culture mediums to conduct proliferation culture to the human Vγ9Vδ2T cells; and IL2, IL2+VC, IL2+IL15, IL2+IL15+VC in FIG. 3 represented the same meanings with those in FIG. 1 and FIG. 2.

Based on FIG. 3, it could be seen that the combination of IL-15 and vitamin C could make the critical killer molecule NKG2D of human Vγ9Vδ2T cells continuously maintain a high level of expression, and the critical killer molecule NKG2D could still maintain a high level of expression at the 21^{st} day of the culturing and after 21 days of culturing. In other words, the formula of the combination of IL-15 and VC (i.e., the technical solution of the present disclosure) could maintain a longer time of the high killing ability of human Vγ9Vδ2T cells.

### Example 2: Anti-Tumor Effect of the Proliferated Vγ9Vδ2T Cells

### Construction of Humanized Mouse Lung Cancer Model and Experimental Methods

### 1. Experimental Animals

Rag2^{-/-}γc^{-/-} female mice aged 3-4 weeks were purchased from Taconic Company, which were of SPF grade. Immunodeficient mice used independently ventilated IVC cages. 5 mice were raised in each cage. Each isolator had independent HEPA air inlet and outlet and had 24-hour temperature and humidity control, and the feed and litter supply of the mice were vacuum-packed and sterilized by γ-radiation, and sterilized water was used as animal drinking water, and the animal raised in this environment was also packaged in a sterile environment; and the animals were transported in a biologically safe transportation box to ensure that the feeding and transportation were maintained in the same microbial state, so as to ensure and maintain the quality of the animals. The animal experiment was approved by the experimental animal ethics committee.

### 2. Method of Constructing Animal Models with Tumors

PBMC (huPBMC) were separated by Ficoll liquid density gradient centrifugation to construct humanized mouse models (huPBMCs humanized mice). The HLA type of constructing the humanized mouse-related PBMC was not consistent with the γδ T cells for reinfusion, and generally was HLA-A2+/−. It was convenient for distinguishing the γδ T cells for reinfusion and from the humanized mice themselves during subsequent detection.

Then, normal Rag2^{-/-}γc^{-/-} mice aged 4-6 weeks were selected, after being irradiated with a sublethal dose of 300 cGay, 30×10⁶ huPBMCs were injected intraperitoneally. After 4 weeks, the surviving humanized mice could be used for the construction of lung cancer models in the next step. After A549 cells growing adherently were digested, a cell suspension was prepared, and the concentration of A549 cells was adjusted to 1×10⁷ cells/mL with phosphate buffered saline (PBS). The prepared cell suspension was inoculated into humanized mice by inguinal subcutaneous injection at a dose of 100 μL/mouse.

### 3. Use of Proliferated Vγ9Vδ2T Cells to Treat Animal Models

1) After 5-7 days of tumor formation, 5×10⁶ γδ T cells cultured by IL-2, IL-2+VC, IL-2+IL-15, IL-2+II-15+VC were injected into the humanized mouse model with lung cancer through the tail vein of the mouse every 3 days, and there were 5 mice in each group, and the dose is 100 μL/mouse. The PBS injection group was a control group of treatment. The γδ T cells cultured by IL-2, IL-2+VC, IL-2+IL-15 and IL-2+II-15+VC were obtained by the method and steps described in the portion of “Vγ9Vδ2T cell proliferation in vitro” in embodiment 1, and IL2, IL2+VC, IL2+IL15 and IL2+IL15+VC represented the same meanings with those in FIG. 1 in embodiment 1.
2) After 3-4 times of treatment, they were observed for more than 60 days. The size of the tumor of the mice was measured using a vernier caliper twice a week, wherein the short diameter (A) and long diameter (B) of the tumor were used to calculate the tumor volume according to the formula: V=½×A²×B.
3) After the reinfusion course of treatment was ended, the peripheral blood was drawn to detect the percentage and cell activity of the γδ T cells in the mice, and after the reinfusion was ended, the peripheral blood was drawn every two weeks to detect the percentage and cell activity of γδ T cells: NKG2D, PDI, CD107a, Fas and FasL.
4) Before the mice were sacrificed, the colonization situation of γδ cells in the mice was detected. GFP signal was detected at the same time, and the number and percentage of A549 tumor cells in peripheral blood were observed.

### 4. Experimental Result

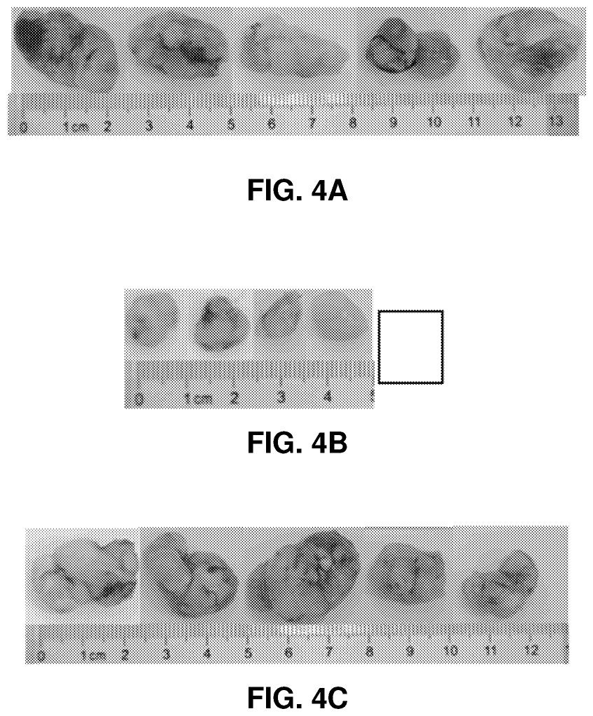

The result of tumor treatment experiment was shown in FIGS. 4A, 4B, 4C, 5 and 6.

FIG. 4A was a schematic view of the size of tumor after a cell treatment to a humanized mouse with lung cancer using the human Vγ9Vδ2T cells obtained by a conventional proliferation method (IL-2).

The conventional proliferation method (the second type of proliferating technology) was always to add IL2 into the basic culture medium for culturing and to add ZOL for stimulating proliferation in the early stage of the culturing.

FIG. 4B was a schematic view of the size of tumor after a cell treatment to a humanized mouse with lung cancer using the human Vγ9Vδ2T cells obtained by the proliferation method (IL2+IL15+VC) of the present disclosure.

FIG. 4C was a schematic view of the size of tumor of the humanized mouse with lung cancer in the case of not being treated.

The humanized mice with lung cancer used in the experiments as illustrated in FIGS. 4A, 4B and 4C had the same tumor type and substantively the same tumor volume, and growing time of the tumor as illustrated in FIGS. 4A, 4B and 4C were the same.

Through the comparison of FIGS. 4A, 4B and 4C, it could be seen that after the human Vγ9Vδ2T cells obtained by the proliferation method of the present disclosure were used to perform cell treatment on the humanized mice with lung cancer, compared with the human Vγ9Vδ2T cells obtained by a conventional proliferation method, the human Vγ9Vδ2T cells proliferated by the present disclosure could more effectively suppress the growth of lung cancer cells (the size of the tumor was significantly reduced), and could significantly improve the survival rate of humanized mice with lung cancer. On the 42^{nd} day, the humanized mice with lung cancer that were treated with human Vγ9Vδ2T cells obtained by a conventional proliferation method and the untreated humanized lung cancer mice all died, however, the humanized mice with lung cancer that were treated with human Vγ9Vδ2T cells obtained by the proliferation method of the present disclosure all survived with a survival rate of 100%, and the tumor in one mouse completely disappeared (shown in the block of FIG. 4B).

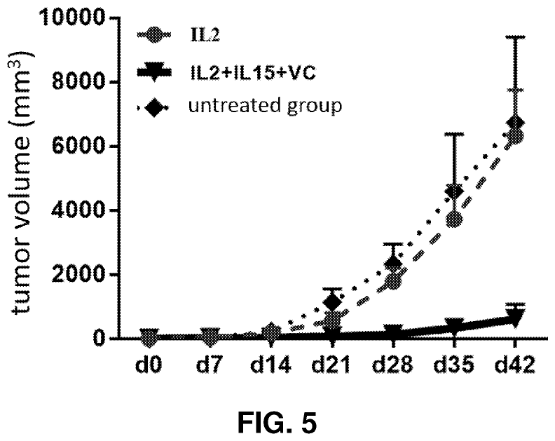

FIG. 5 was a schematic view of the variations of the volume of the tumor of the humanized mice with lung cancer under different treating conditions, and FIG. 5 respectively illustrated the volume changes of the tumor of the humanized mice with lung cancer when the humanized mice with lung cancer were treated with the human Vγ9Vδ2T cells obtained by the conventional proliferation method (IL2) and with the human Vγ9Vδ2T cells obtained by the proliferation method (IL2+IL15+VC) of the present disclosure, and were not treated.

It could be seen that with the increase of time, the volumes of tumors of the humanized mice with lung cancer which were treated with human Vγ9Vδ2T cells obtained by the conventional proliferation method (IL2) and which were not treated increased continuously, and the tumor volumes increased very quickly in the later stage, however, the tumor volume of humanized mice with lung cancer treated with the human Vγ9Vδ2T cells obtained by the proliferation method (IL2+IL15+VC) of the present disclosure increased very slowly, in other words, compared with the human Vγ9Vδ2T cells obtained by the conventional proliferation method (IL2), the human Vγ9Vδ2T cells obtained by the proliferation method (IL2+IL15+VC) of the present disclosure had a stronger suppressing effect on tumor growth in vivo, and rendered smaller tumor volumes.

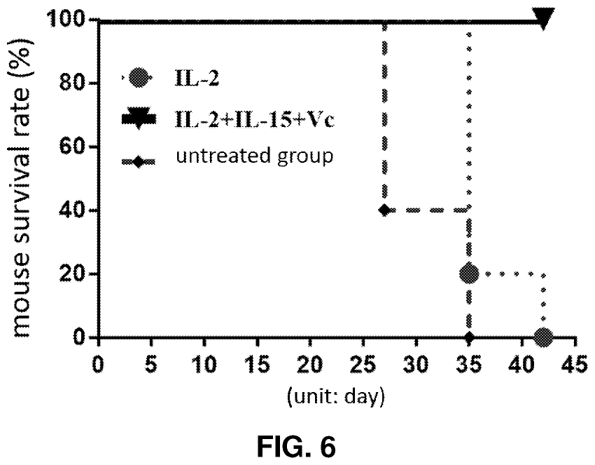

FIG. 6 was a schematic view of the variations of the survival rates of the humanized mice with lung cancer under different treating conditions, and FIG. 6 respectively illustrated the variations of the survival rates of the humanized mice with lung cancer which were treated with the human Vγ9Vδ2T cells obtained by the conventional proliferation method (IL2) and with the human Vγ9Vδ2T cells obtained by the proliferation method (IL2+IL15+VC) of the present disclosure, and which were not treated.

It could be seen that with the increase of time, the survival rates of the humanized mice with lung cancer which were treated with human Vγ9Vδ2T cells obtained by the conventional proliferation method (IL2) and which were not treated reduced very quickly, however, the survival rate of the humanized mice with lung cancer treated with the human Vγ9Vδ2T cells obtained by the proliferation method (IL2+IL15+VC) of the present disclosure was always 100%, in other words, the human Vγ9Vδ2T cells obtained by the proliferation method (IL2+IL15+VC) of the present disclosure could significantly improve the survival rate of the humanized mouse with lung cancer.

### Example 3

Example 3 provided a culture medium, which comprised a RPMI-1640 culture medium, and interleukin-2, interleukin-15 and vitamin C added in the RPMI-1640 culture medium.

In the above, in the culture medium, the concentration of the interleukin-2 was 300 ng/ml, the concentration of the interleukin-15 was 500 ng/ml, and the concentration of the vitamin C was 100 mM.

### Example 4

Example 4 provided a culture medium, which comprised a RPMI-1640 culture medium, and interleukin-2, interleukin-15 and vitamin C added in the RPMI-1640 culture medium.

In the above, in the culture medium, the concentration of the interleukin-2 was 500 ng/ml, the concentration of the interleukin-15 was 700 ng/ml, and the concentration of the vitamin C was 300 mM.

### Example 5

Example 5 provided a culture medium, which comprised the second culture medium as mentioned in the Example 1 and zoledronic acid.

In the above, in the culture medium, the concentration of the zoledronic acid was 300 μM.

### Example 6

Example 6 provided a culture medium, which comprised the second culture medium as mentioned in the Example 4 and zoledronic acid.

In the above, in the first culture medium, the concentration of the zoledronic acid was 500M.

### Example 7

Between July and December 2018, eight patients with MDR-TB (Multidrug-Resistant Tuberculosis) diagnosed using M. tuberculosis sputum smear microscopic examination, culture, and drug sensitivity testing were recruited in our study after signing an informed consent. The age of the patients ranged between 18 and 50 years old. Patients with other conditions, including HIV, hepatitis B, diabetes, tumor, hypertension, coronary heart disease, endocrine system disease, mental disease, neurological disease, vascular circulation system disease, or other disease, were excluded from our study.

The Vγ9Vδ2T cells cultured were obtained by the method described in Example 1. We adoptively infused the expanded cells into the enrolled patients. In our protocol, patients received a single treatment of 1×10⁸ Vγ9Vδ2 T cells every half month with a total of 12 courses completed in 6 months, followed by a 6-month follow-up.

As the M. tuberculosis load is a key indicator for evaluating therapeutic efficacy, we performed sputum M. tuberculosis detection in all patients (see Table 2) We observed that M. tuberculosis was detected in the sputum of patients regardless of the methods used (sputum smear acid-fast bacillus testing or sputum culture testing) before Vγ9Vδ2 T-cell therapy. After all patients had finished therapy, patient #2, who had severe MDR-TB, still had a M. tuberculosis-positive sputum culture. However, the lesions of patient #2 were significantly decreased. The sputum M. tuberculosis turned to be negative 3 times consecutively in the remaining seven patients. According to the criteria of the World Health Organization, a 3 times consecutive negative detection of sputum M. tuberculosis indicates complete clinical control of M. tuberculosis infection. The results demonstrate that the Vγ9Vδ2 T-cell of the present disclosure contribute a lot in alleviating the M. tuberculosis load in vivo.

TABLE 2
Sputum Mycobacterium tuberculosis examination of all patients
before and after Vγ9Vδ2 T-cell immunotherapy.

| | | #1 | #2 | #3 | #4 | #5 | #6 | #7 | #8 |
|---|---|---|---|---|---|---|---|---|---|
| Sputum smear | Before | − | + | − | + | − | + | + | + |
| (acid-fast bacillus) | | | | | | | | | |
| Sputum | Before | + | − | + | + | + | + | + | + |
| M. tuberculosis culture | | | | | | | | | |
| Sputum smear | After | − | − | − | − | − | − | − | − |
| (acid-fast bacillus) | | | | | | | | | |
| Sputum | After | − | + | − | − | − | − | − | − |
| M. tuberculosis culture | | | | | | | | | |

### Example 8

A 30-year-old man was diagnosed as Cholangiocarcinoma with mediastinal lymph node metastasis stage IV. In July 2013, he received treatment at a local hospital for Crohn's disease. In November 2014, he received a liver transplantation; a huge tumor at hepatic portal was intraoperatively resected. The postoperative pathology report revealed a liver and hepatic portal poorly-differentiated adenocarcinoma with unresectable Cholangiocarcinoma metastasized to lymph nodes.

The MRI scan performed on Feb. 24, 2015 showed a lesion in patient's liver, therefore, he received lymph node resection on Apr. 13, 2015. From Jun. 13, 2015 to Aug. 14, 2015, the patient received radiotherapy for hepatic portal and the area adjacent to inferior vena cava, with a total dosage of 45 Gy. Afterward, the patient did not receive any further anti-cancer treatments, except follow-up visits. The PET/CT collected on Apr. 15, 2016, showed lesions in mediastinum and liver. On Jun. 29, 2016, the patient came to the Fuda Cancer Hospital. Firstly, aspiration biopsy was conducted and 10 I125 was seeded into the mediastinal tumor. On June 2017, when the patient came back to Fuda Cancer Hospital for follow-up check-up, biopsy result showed recurrent abdominal lymph node metastasis by experts' consultation, therefore starting from June 2017, the patient only received γδ T cell immunotherapy to control his lesions, and the first γδ T cell infusion was scheduled on August 2017.

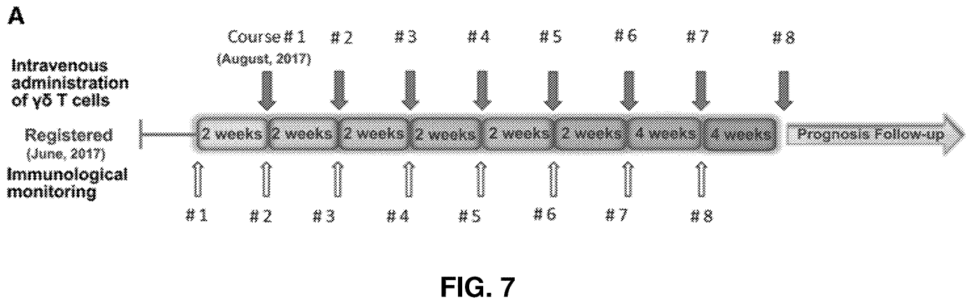

The Vγ9Vδ2T cells cultured were obtained by the method described in Example 1. About 4×10⁸ Vγ9Vδ2 T cells of the present disclosure were infused into the patient. FIG. 7 indicates the schedule of Vγ9Vδ2 T cell treatments.

As shown in FIG. 7, the patient was enrolled in on June 2017, and received cell treatments starting from August 2017. The patient received 8 treatment courses (3 infusions per treatment course infused within 2 days) of Vγ9Vδ2 T cell treatments from August 2017 through February 2018. Infusion was performed every 2 weeks for first six infusions, and then 4 weeks for last two infusions.

During Vγ9Vδ2 T cell treatment, tumor was routinely evaluated by using MRI imaging to monitor tumor size/area changes by the largest transverse diameter, particularly before and after treatment. The patient received plain and enhanced MRI 2 weeks before treatment, and then scanned periodically at the 3rd and 6th months after treatment.

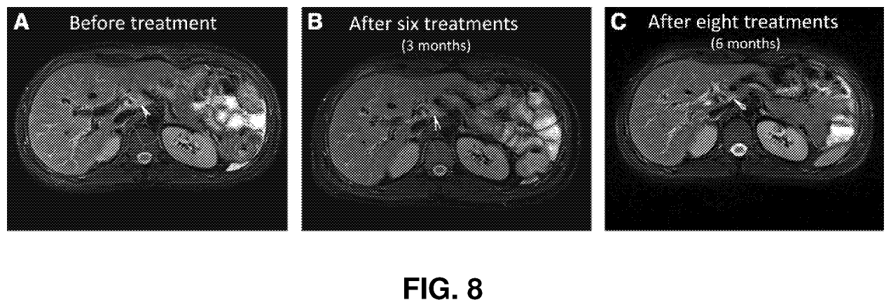

In FIG. 8, upper abdominal MRI examinations were taken at 3 time points, a 2 weeks before treatment, b 3 month's clinical effect post treatment and c 6 months clinical effect post treatment. In this figure, representative MRI images obtained before entry into the clinical trial and after the 8th treatment course are displayed.

From the MRI images (FIG. 8), we can see that the size of the lymph nodes is markedly reduced, visualizing that lymph nodes metastases of the patient were gradually eliminated with increasing infusion times of Vδ2 T cells. Such visual images indicated that the patient greatly benefited from the Vγ9Vδ2 T cell treatment in this case.

### Example 9

A clinical trial in 132 late-stage cancer patients with Vγ9Vδ2 T cell infusions unequivocally validated the clinical safety the efficacy of Vγ9Vδ2 T cell of the present disclosure, among which 8 liver cancer patients and 10 lung cancer patients received ≥5 cell infusions.

The Vγ9Vδ2T cells cultured were obtained by the method described in Example 1. About 1-2×10⁸ Vγ9Vδ2 T cells of the present disclosure were infused into the patients each time. Patients received cell therapy once every 2-3 weeks for the first five infusions, then once per month or every 2 months.

In this trail, only 8 liver cancer and 10 lung cancer patients received ≥5 Vγ9Vδ2 T-cell infusions, and these 18 patients were then followed.

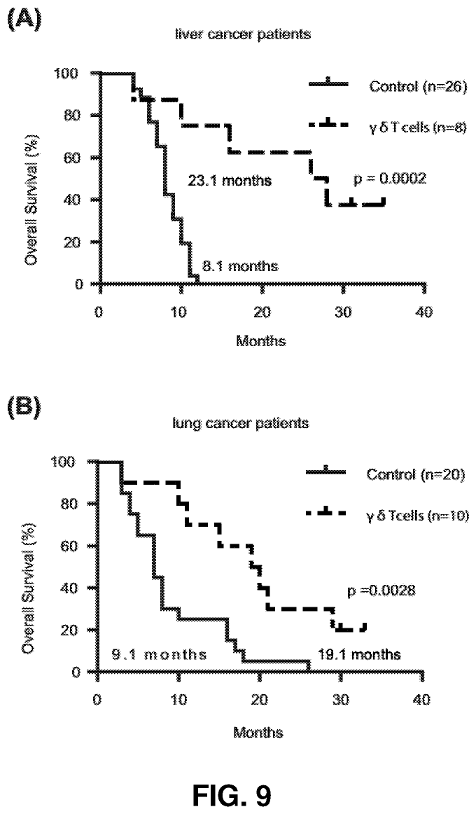

The survival data are shown in FIG. 9.

For the liver cancer group, the median survival time of the untreated patients was 8.1 months; in contrast, the median survival time of the Vγ9Vδ2 T-cell-treated patients was 23.1 months (P=0.0002). For the lung cancer group, the median survival time of the untreated patients was 9.1 months; however, the median survival time was 19.1 months for the γδ T-cell-treated patients (P=0.0028).

As shown in FIG. 9, seven of the eight liver cancer patients (FIG. 9A) and nine of the ten lung cancer patients (FIG. 9B) survived ≥10 months. In our latest follow-up, the results showed that three liver cancer and two lung cancer patients were still alive between 30 and 35 months after the Vγ9Vδ2 T-cell therapy (see gray columns in FIG. 9B).

### Example 10

### Tumor Cell Killing Experiment (CFSE/PI Method)

1) Tumor cells were collected, centrifuged, and then resuspended with 2 mL serum-free medium. 100 μL was reserved as a blank, and 100 μL was reserved for PI staining as a single-stained tube.
2) The cells were washed 2-3 times with 3-4 mL of sterile PBS to remove serum.
3) 1 mL of the resuspended tumor cells were transferred into a 1.5 mL EP tube. 0.4 μL of CFSE (concentration 5 mM) was added to achieve a final concentration of 2 μm, followed by incubation at 37° C. for 10 min. The remaining cells, which were not stained with CFSE, were returned to the culture dish.
4) After the staining was completed, 3 volumes of pre-chilled medium containing serum was added. The mixture was placed on ice for 5 min to terminate the staining. The cells were then washed twice with PBS, resuspended by adding medium, and counted. A portion of the CFSE-stained tumor cells were reserved as a CFSE single-stained tube.
5) Vγ9Vδ2 T cells were collected, resuspended, and counted (a portion was added to the blank in step 1).
6) 1×10⁵ tumor cells were taken. 3 control groups were set up without Vγ9Vδ2 T cells but only with CFSE-stained tumor cells. Subsequently, different effector-to-target ratios of 1:1, 5:1, 10:1, and 20:1 were established for co-culture in round-bottom flow tubes.
7) Final medium volume was 150 UL at co-culture.
8) Co-culture was carried out for 6 hours. The cells were harvested, washed once, stained with PI, and analyzed by flow cytometry. Cells that were double-positive for CFSE and PI were identified as killed target cells.

Herein, the specific cytotoxic activity is calculated using the following formula:

specific cytotoxic activity (%)=(mortality of target cells in experimental group-spontaneous mortality of target cells)/(100−spontaneous mortality of target cells)*100%.

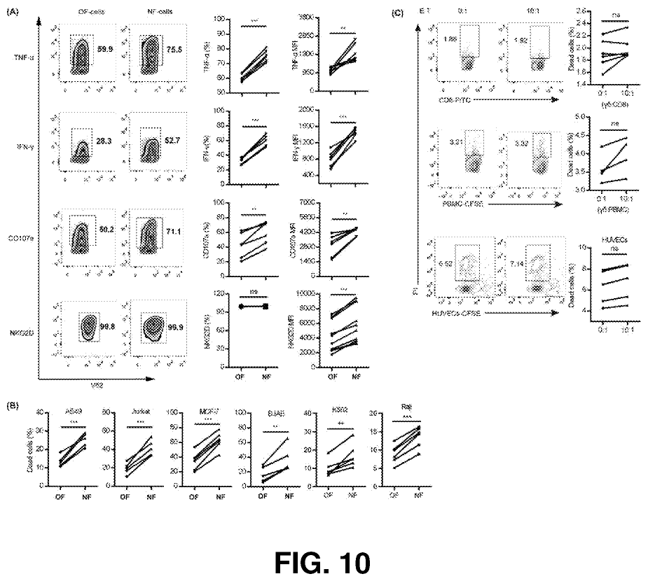

To further explore the effect of IL-2, IL-15, and Vc on the killing molecules of Vγ9Vδ2 T cells, the present disclosure employed the flow cytometry to detect the expression of the intracellular killing molecules of Vγ9Vδ2 T cells cultured for 12-14 days with different cytokines in vitro. As shown in FIG. 10A, the novel formulation of the present disclosure greatly improved the expression of effector molecules (IFN-γ, TNF-α, and NKG2D) and degranulation molecule CD107a. In addition, the present disclosure further investigated the in vitro killing ability against normal cells and various types of cancer cells (lung cancer cell line A549, acute T-cell leukemia cell line Jurkat, breast cancer cell line MCF-7, human Burkitt lymphoma cell line BJAB, chronic myeloid leukemia cell line K562, and human lymphoma cell line Raji). As shown in FIG. 10C, NF cells exhibited no cytotoxicity towards normal cells, such as CD8 T cells, PBMCs, and human umbilical vein endothelial cells (HUVECs). As shown in FIG. 10B, NF cells exhibited significantly enhanced anti-tumor cytotoxicity in vitro against cancer cell lines.

Upon stimulation of Vγ9Vδ2 T cells with anti-CD3 (5 μg/mL)/CD28 (1 μg/mL) and Golgistop (1:1000) for 6 h, the contents of intracellular TNF-α, IFN-γ, CD107a, and NKG2D were detected by flow cytometry (n=6) (see FIG. 10A). Vγ9Vδ2 T cells cultured in vitro for approximately 12-14 days with purity higher than 85% were washed twice with serum-free RPMI-1640 to completely remove various cytokines. Tumor cells were labeled with CFSE, and well mixed with the above tumor cell lines and normal cells at a certain ratio of lymphocyte effector cells to target cells of 10×10⁵:1×10⁵ (10:1). The mixture was added to a U-bottom 96-well plate. After 6 hours of co-culture, the cells were stained with PI prior to flow cytometric analysis. The killing rate of the Vγ9Vδ2 T cells against tumor cells was determined by flow cytometric analysis of the percentage of CFSE+PI+ cells among viable target cells.

TABLE 3
Effector-to-Target Ratios for γδ T Cell Killing of HepG2 Cells

| | Effector- | | | | | |
|---|---|---|---|---|---|---|
| | to-Target | | | | | |
| | Ratio | | | | | |
| Cell | (E:T) | Donor 1 | Donor 2 | Donor 3 | Donor 4 | Donor 5 |
| HepG2 | 1:1 | 19.57 | 21.41 | 21.41 | 33.40 | 37.00 |
| | 5:1 | 9.58 | 38.84 | 46.02 | 58.90 | 44.60 |
| | 10:1 | 27.52 | 63.15 | 44.19 | 63.10 | 50.00 |
| | 20:1 | 56.12 | 48.62 | 44.80 | 65.40 | 61.70 |

TABLE 4
Effector-to-Target Ratios for γδ T Cell Killing of Huh7 Cells

| | Effector- | | | | | |
|---|---|---|---|---|---|---|
| | to-Target | | | | | |
| | Ratio | | | | | |
| Cell | (E:T) | Donor 1 | Donor 2 | Donor 3 | Donor 4 | Donor 5 |
| Huh7 | 1:1 | 5.10 | 8.40 | 7.90 | 8.30 | 32.70 |
| | 5:1 | 34.70 | 32.10 | 22.40 | 45.20 | 55.50 |
| | 10:1 | 42.30 | 36.70 | 33.60 | 50.10 | 58.60 |
| | 20:1 | 50.60 | 44.30 | 36.30 | 47.10 | 60.80 |

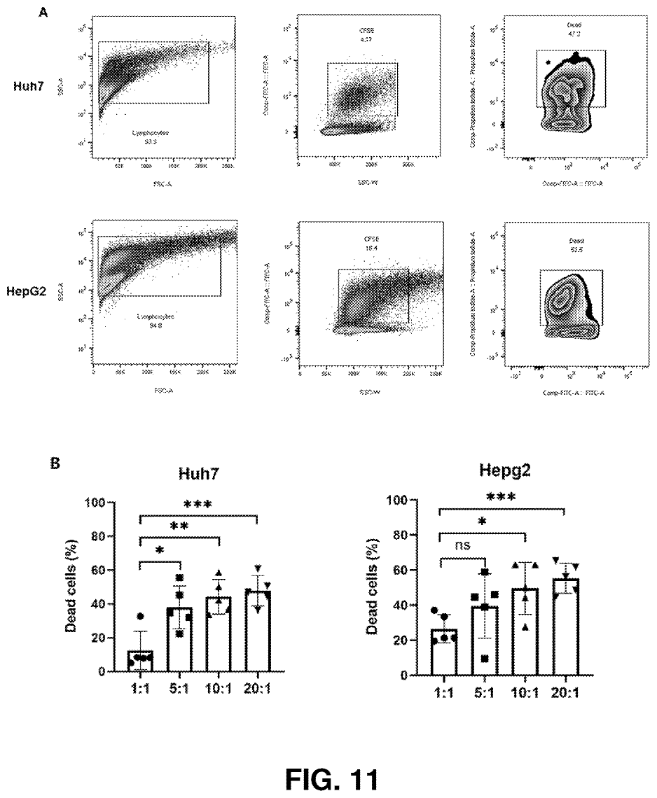

As shown in Table 3, Table 4, and FIG. 11, the results of the in vitro killing experiment demonstrated that the Vγ9Vδ2 T cells of the present disclosure are capable of efficiently killing the liver cancer cell lines Huh7 and HepG2 cells.

### Example 11

### Construction of Liver Cancer Model in Immunodeficient Mice (CDX)

20 α-NDG mice, aged 3-4 weeks with an average body weight of 18-22 g, were selected. The mice, half male and half female, were purchased from Biocytogen Pharmaceuticals (Beijing) Co., Ltd. and were of SPF grade. The animal experiments were approved by the experimental animal ethics committee.

### 1) HepG2 Cell Recovery, Culture, and Cryopreservation

Preparation of 10% DMEM medium (500 mL): To 450 mL of serum-free DMEM medium, 5 mL of fetal bovine serum and double antibiotics (penicillin-streptomycin) at a final concentration of 1% were added sequentially, thoroughly mixed, sealed with a sealing film, and stored at 4° C. for later use.

Cell recovery: The constant-temperature water bath was pre-heated to 37° C. HepG2 cells were quickly retrieved from the liquid nitrogen tank and placed in the constant-temperature water bath, where they were rapidly shaken back and forth until completely thawed. Following thawing, the cells were transferred to a 15 mL centrifuge tube, and an appropriate amount of serum-free DMEM medium was added. The cells were centrifuged at room temperature for 5 min at 1000 rpm to remove the cryopreservation solution DMSO. After centrifugation, the supernatant was discarded, and residual liquid was drained off. The cell pellet was resuspended in freshly prepared 10% DMEM medium, transferred to a T25 or T75 cell culture flask, and incubated at 37° C. in an incubator with 5% CO₂.

Cell passaging: The cells were observed under an inverted microscope. When the cell growth density reached 80%-90% confluence, the old culture medium was discarded. The culture flask was washed 2-3 times with serum-free DMEM medium, followed by digestion with 1-2 mL of trypsin (0.25%) 37° C. for 1-2 min. After digestion, 5-10 volumes of 10% DMEM medium was added to terminate the digestion. The digestion mixture was then transferred into a 15 mL or 50 mL centrifuge tube and centrifuged at room temperature for 5 min at 1000 rpm. The supernatant was discarded, and the pellet was resuspended in an appropriate amount of 10% DMEM medium. After thorough mixing, the cells were seeded back into T75 cell culture flasks at a ratio of 1:2 or 1:3 and further cultured in a 37° C. incubator with 5% CO₂.

Cell cryopreservation: The initial steps were the same as those for the cell passaging operation. After centrifugation, the cell pellet was collected, resuspended in the cell cryopreservation solution (the volume ratio of fetal bovine serum to DMSO was 9:1, prepared just before use), mixed uniformly and then aliquoted into cell cryopreservation tubes at 1 mL per tube. After labeling (cell type, date, etc.), the tubes were placed into a freezing container and transferred to a −80° C. freezer overnight. The following day, the tubes were transferred to a liquid nitrogen tank for long-term storage.

### 2) Tumor Cell Suspension Preparation

HepG2 cells in the exponential phase and in a good growth state were collected. The medium was removed by centrifugation. The cells were washed once by resuspension in sterile NaCl injection. Subsequently, the cells were resuspended in serum-free DMEM medium, and counted, and the cell density was adjusted to 5×10⁷ cells/mL. The cells were prepared according to the number of mice and the cell amount required for inoculation. The cell suspension was aliquoted into sterile 1.5 mL EP tubes or 50 mL centrifuge tubes and sealed with a sealing film. The tumor cell suspension was then injected subcutaneously into the inguinal region of left hind limb of the mice, at 0.1 mL per mouse. After inoculation, the mice were observed daily until the mice developed tumors.

### 3) In Vivo Reinfusion of Vγ9Vδ2 T Cells for Treatment

Vγ9Vδ2 T cells from D11 or D13 in Example 1 were collected into a 50 mL centrifuge tube, and centrifuged for 5 minutes at 800-1000 rpm. The supernatant was discarded. 1-2 mL of NaCl injection was added. After thorough pipetting, another 20-30 mL of NaCl injection was added, followed by centrifugation at 800-1000 rpm for 5 minutes. The cells were washed 2-3 times. Depending on the cell pellet, the cells were resuspended in 1-2 mL of NaCl injection, diluted to an appropriate fold (100-200), and counted. The γδ-T cell density was adjusted with the NaCl injection. After thorough pipetting, the prepared Vγ9Vδ2 T cells were aliquoted into corresponding sterile 1.5 mL EP tubes or 50 mL centrifuge tubes based on the required total number of γδ-T cells, the number of animals to be inoculated, and the inoculation volume. The tubes were sealed with a sealing film and kept on ice. Each mouse in the experimental group received a reinfusion of 2×10⁷ cells in 100 μL once weekly for a total of 5 doses. Each mouse in the control group received a reinfusion of 100 UL of NaCl solution once weekly for a total of 5 doses.

### 4) HE Staining

After the experiment was ended, the mice were subjected to necropsy, and tumor, heart, liver, spleen, lung, kidney and other organs thereof were collected, fixed in 4% paraformaldehyde fix solution for 1-2 days, and subsequently subjected to hematoxylin-eosin staining (HE staining).

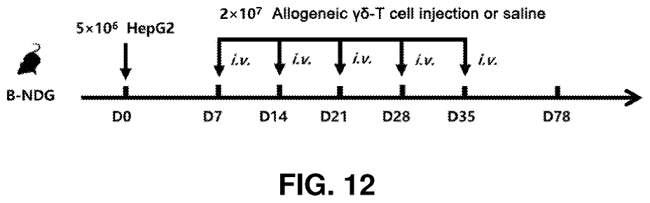

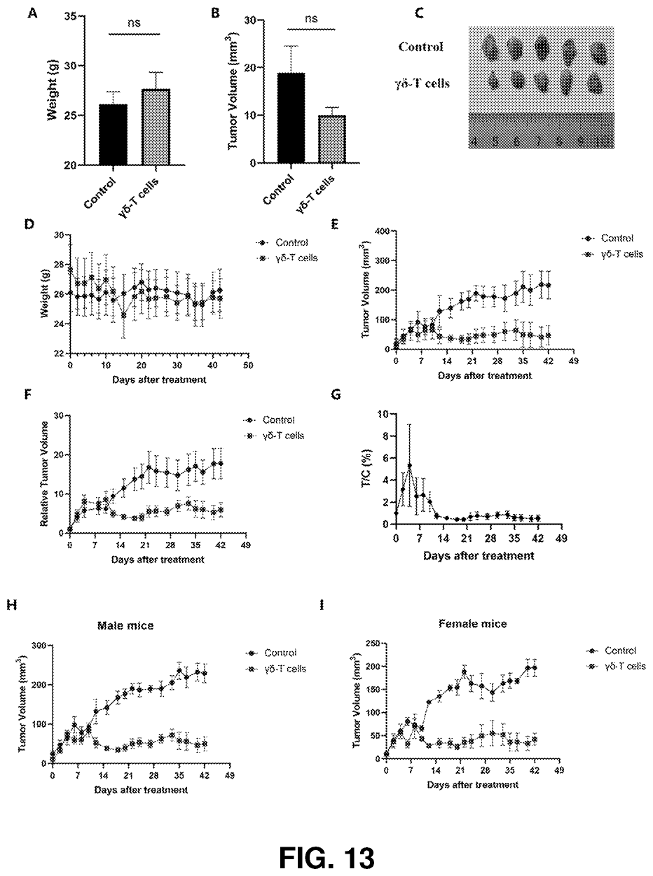

FIG. 12 indicates the schedule of Vγ9Vδ2 T cell reinfusion treatments. Prior to administration of the Vγ9Vδ2 T cell therapy of the present disclosure, the tumor-bearing mice were weighed, and the tumor volumes were measured. Subsequently, the mice were randomly divided into two groups (control group and treatment group). As shown in FIG. 13, at the time of grouping, there were no significant differences in body weight or tumor volume between the mice in the control group and the mice in the treatment group (FIGS. 13A and 13B). The results from the liver cancer-bearing mouse models demonstrated that the tumor volumes of the mice in the treatment group were significantly reduced compared with those in the control group (FIG. 13C); there was no significant difference in mouse body weight between the treatment group and the control group (FIG. 13D); tumors of the mice in the treatment group showed a declining trend after the second reinfusion of Vγ9Vδ2 T cells and maintained a stable state thereafter (FIGS. 13E and 13F); the relative tumor growth inhibition rate curve also exhibited a declining trend after the second reinfusion of Vγ9Vδ2 T cells and subsequently maintained a stable state, indicating that in vivo reinfusion of Vγ9Vδ2 T cells could remarkably control the tumor progression in mice (FIG. 13G). The above results collectively demonstrate that the in vivo reinfusion of Vγ9Vδ2 T cells can effectively control the tumor initiation and progression in mice. Furthermore, we also analyzed the tumor sizes separately for female and male mice to evaluate the variability of in vivo reinfusion therapy with Vγ9Vδ2 T cells between the female and male mice. The statistical results revealed that the Vγ9Vδ2 T cells of the present disclosure exerted positive therapeutic effects in both female and male mice, effectively controlling tumor cell initiation and progression in mice (FIGS. 13H and 131).

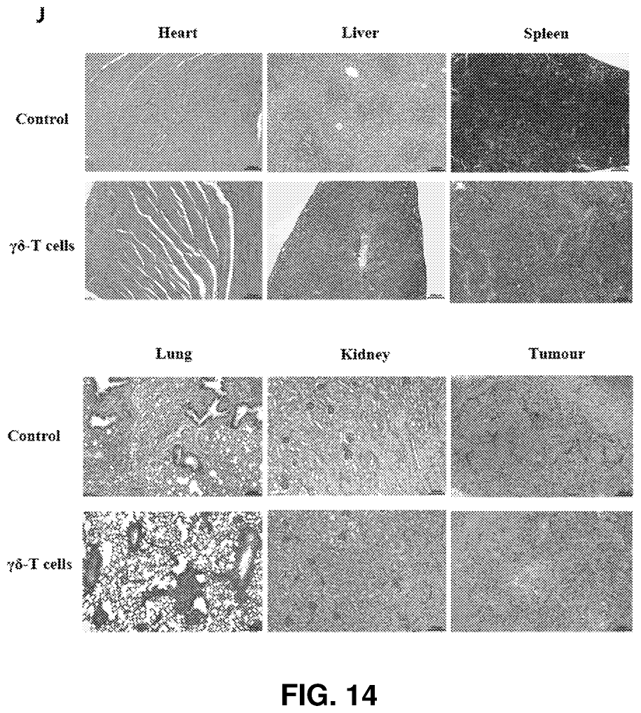

Since the Vγ9Vδ2 T cells used for reinfusion were derived from different healthy volunteers, to further verify that the reinfused Vγ9Vδ2 T cells will not cause damage to the organs of the mice, we euthanized the liver cancer-bearing mice and collected the heart, liver, spleen, lung, kidney, and tumor of the mice for HE staining. The results demonstrated that in the mice in the control group, no significant abnormalities were observed in the heart, liver, spleen, lung, or kidney, while the tumors exhibited severe proliferation with visible mitotic figures. In the mice in the treatment group, no significant abnormalities were observed in the heart, liver, spleen, lung, or kidney either, but the tumors exhibited mild proliferation with visible mitotic figures (FIG. 14). That is, compared with the control group, the degree of tumor cell proliferation was reduced in the experimental group, indicating that the administration of reinfusion therapy with Vγ9Vδ2 T cells can inhibit the proliferation and progression of HepG2 tumor cells to a certain extent. In summary, the in vivo reinfusion of the Vγ9Vδ2 T cells does not cause substantial adverse effects on the organs of mice. Moreover, based on the degree of tumor cell proliferation, the reinfusion of the Vγ9Vδ2 T cells can effectively inhibit or attenuate the proliferation of HepG2 tumor cells to a certain extent.

In the mouse models of liver cancer, the allogeneic Vγ9Vδ2 T cells of the present disclosure exhibit potent anti-tumor efficacy, significantly inhibiting the tumor growth in mice and prolonging the survival. Meanwhile, the safety and efficacy of the allogeneic Vγ9Vδ2 T cell reinfusion therapy were validated. It is difficult to describe all the numerical ranges of the process parameters involved in the present disclosure in the above-mentioned embodiments, but those skilled in the art can fully imagine that any value falling within the above-mentioned value range can perform the present disclosure, and certainly the present disclosure contains any combination of specific values of several numerical ranges. Here, for the sake of space, the embodiments providing specific values within one or more numerical ranges are omitted, while this should not be regarded as insufficient disclosure of the technical solutions of the present disclosure.

The applicant declares that the above-mentioned embodiments in the present disclosure are used to describe the specific process equipment and the process flow of the present disclosure, but the present disclosure are not limited to the above process equipment and process flow, that is, it does not mean that the present disclosure can only be implemented depending on the above specific process equipment and process flow. A person skilled in the art should understand that any improvement to the present disclosure, and equivalent substitutions to each material of the product of the present disclosure, and the addition, the selection of the specific methods of auxiliary components and the like all fall within the scope of the present disclosure.

### INDUSTRIAL APPLICABILITY

Compared with a conventional proliferation method (the second type of proliferation technology), the human Vγ9Vδ2T cells obtained by the proliferation method of the present disclosure has a higher proliferation efficiency and a higher cell purity, and the proliferation method of the present disclosure obviously shortens the proliferation culture period of the human Vγ9Vδ2T cells. Compared with the human Vγ9Vδ2T cells obtained by the conventional proliferation method (the second type of proliferating technology), the human Vγ9Vδ2T cells obtained by the proliferation method of the present disclosure have stronger killing and suppressing abilities to tumor. The human Vγ9Vδ2T cells obtained by the proliferation method of the present disclosure have a stronger anti-apoptotic ability and a longer survival time.

What is claimed is:

1. A T cell proliferation method, comprising:
step A, culturing a composition containing the T cells, by using a first culture medium which is able to stimulate T cells, so as to stimulate the T cells; and
step B, using a second culture medium to culture stimulated T cells, wherein the second culture medium contains interleukin-2, interleukin-15, and vitamin C or a derivative of vitamin C.

2. The method according to claim 1, wherein the T cells comprise Vγ9Vδ2T cells.

3. The method according to claim 1, wherein the first culture medium comprises interleukin-2 and phosphonic acid compounds, and optionally, also comprises interleukin-15, and vitamin C or a derivative of vitamin C.

4. The method according to claim 1, wherein the derivative of vitamin C is selected from the group consisting of vitamin C ethyl ether, vitamin C palmitate, vitamin C glucoside, vitamin C magnesium phosphate and vitamin C sodium phosphate and combinations thereof.

5. The method according to claim 1, wherein in the second culture medium, a concentration of the interleukin-15 is 1-1000 ng/ml.

6. The method according to claim 1, wherein in the second culture medium, a concentration of the vitamin C or the derivative of vitamin C is 10 μM-800 mM.

7. The method according to claim 1, wherein in the second culture medium, a concentration of the interleukin-2 is 1-1000 ng/ml.

8. The method according to claim 1, wherein the composition containing the T cells comprises peripheral blood mononuclear cells extracted from peripheral blood of human body.

9. The method according to claim 1, wherein in the step A, a culture duration is 60-100 hours.

10. The method according to claim 3, wherein the phosphonic acid compounds comprise bisphosphonic acid compounds.

11. The method according to claim 1, wherein the second culture medium further comprises a basic culture medium, wherein the basic culture medium is selected from the group consisting of RPMI-1640 culture medium, D-MEM, MEM, RPMI, Opti-MEM and combinations thereof, for example, RPMI-1640 culture medium.

12. A culture medium, comprising a basic culture medium, interleukin-2, interleukin-15, and vitamin C or a derivative of vitamin C.

13. The culture medium according to claim 12, wherein a concentration of the interleukin-2 is 1-1000 ng/ml, a concentration of the interleukin-15 is 1-1000 ng/ml, and a concentration of the vitamin C or the derivative of vitamin C is 10 μM-800 mM.

14. A method for suppressing, preventing or treating an infectious disease, an autoimmune disease or a malignant disease, comprising:
(1) extracting peripheral blood mononuclear cells from peripheral blood of a subject in need;
(2) proliferating the peripheral blood mononuclear cells through the method according to claim 1; and
(3) administering proliferated product to the subject in need,
wherein the peripheral blood mononuclear cells are used as the composition containing the T cells, preferably, the malignant disease is cancer.

15. The method according to claim 14, wherein the malignant disease is a solid tumor.

16. The method according to claim 14, wherein the the malignant disease is selected from the group consisting of adrenocortical carcinoma, anal cancer, bladder cancer, ependymoma, medulloblastoma, breast cancer, cervical cancer, colon cancer, endometrial cancer, esophageal cancer, extrahepatic cholangiocarcinoma, eye cancer, gallbladder cancer, gastric cancer, germ cell tumors, extragonadal cancer, head and neck cancer, hypopharyngeal cancer, pancreatic islet cell cancer, laryngeal cancer, leukemia, acute lymphoblastic leukemia, oral cancer, liver cancer, lung cancer, and combinations thereof, for example, lung cancer.

17. The method according to claim 2, wherein the first culture medium comprises interleukin-2 and phosphonic acid compounds, and optionally, also comprises interleukin-15, and vitamin C or a derivative of vitamin C.

18. The method according to claim 2, wherein the derivative of vitamin C is selected from the group consisting of vitamin C ethyl ether, vitamin C palmitate, vitamin C glucoside, vitamin C magnesium phosphate and vitamin C sodium phosphate and combinations thereof.

19. The method according to claim 2, wherein in the second culture medium, a concentration of the vitamin C or the derivative of vitamin C is 10 μM-800 mM.

20. The method according to claim 2, wherein the composition containing the T cells comprises peripheral blood mononuclear cells extracted from peripheral blood of human body.
